(19) United States
(12) Patent Application Publication
(10) Pub. No.: US 2011/0238183 A1
(43) Pub. Date: Sep. 29, 2011

(54) Interbody Fusion Device

(76) Inventor: Richard S. Maly, San Diego, CA (US)

(21) Appl. No.: 12/891,776

(22) Filed: Sep. 27, 2010

Related U.S. Application Data
(60) Provisional application No. 61/277,475, filed on Sep. 26, 2009.

(51) Int. Cl. A61F 2/44

(52) U.S. Cl. 623/17.16

(57) ABSTRACT

An interbody fusion device, or bone dowel for mending of joints is disclosed herein. The device comprises anti-migration ridges having optimum geometry to avoid failure during the insertion process while providing boney ingrowth in the body's natural healing process. A tapered head portion and a cylindrical body without threads are further included herein. Also, the device provides novel means for engagement to an insertion tool. Additional methods are disclosed herein to maintain and insert a device without torsional stresses due to torquing.

### PRIORITY CLAIM

This utility patent application contains subject matter claiming benefit of the priority date of U.S. Provisional Patent Application Ser. No. 61/277,475, filed on Sep. 26, 2009, accordingly, the entire contents of this provisional patent application is hereby expressly incorporated by reference.

### BACKGROUND OF THE INVENTION

1. Field of the Invention

The present invention pertains generally to devices and methods useful in surgical processes that require fusion of joints. More specifically, the present invention is particularly but not exclusively useful as an allograft fusion implant for interbody spinal fusion and related procedures.

2. Description of the Prior Art

Spinal fusion has been known as a procedure improving articular movement and providing stabilization of the spine for sufferers of a variety of aliments including degenerative disc disease, spinal disc herniation, discogenic pain, spinal tumor, vertebral fracture, scoliosis, kyphosis, spondylolisthesis, spondylosis, Posterior Rami Syndrome, and other degenerative spinal conditions. In performing this procedure, implants are applied to an area once occupied by the intervertebral disc between adjacent vertebrae. When the disc is removed, the space between vertebrae endplates will tend to collapse. Accordingly, this space is first reamed to maintain the proper geometry prior to the implant insertion. The implants may also be referred to as dowels, cages, or bone grafts (as the implants are preferably made from allograft material).

Following the implant procedure, the natural osteoblastic process will produce ingrowth from the normal bone healing response. Hence, a high quality allograft bone obtained from organ donors is much preferred over artificial implants. The use of a bone graft presents challenges because they have relatively brittle material properties as compared to plastic or titanium and therefore will fracture more easily under stresses of insertion. Also complicating the problem is that bone from tissue donors is relatively expensive to acquire.

Many proposed designs have been granted U.S. patent protection to include: U.S. Pat. No. 5,593,409, entitled “Interbody Spinal Fusion Implants” to Michelson, U.S. Pat. No. 5,814,084, entitled “Diaphysial Cortical Dowel” to Frivas et al., U.S. Pat. No. 6,371,988 entitled “Bone Grafts” to Pafford et al., and U.S. Pat. No. 6,447,544 entitled “Lordotic Interbody Spinal Fusion Implants” also to Michelson, and U.S. Pub. Pat. App. No. 2010/0057207 entitled “Bone Gratis” to Ray III et al. One problem with many prior solutions is that they provide a tapered dowel along its entire length believing that this is the optimum choice the procedure. Conversely, while many younger healthy spines will have a tapered area between vertebrae, an older spine will more likely have more of a flat area in the disc space. However not so intuitively, the patients needing this procedure are more likely to be older with applicable conditions worsening with age. Also, the ream used to maintain proper space are currently not tapered and thus a tapered design would more likely break during the insertion process. Other improvements to processes and devices for spinal fusion are also needed within the art.

In light of the above, an object of the present invention is to provide an Interbody Fusion Device that has a cylindrical design to closely mirror the space it is to occupy. It is a further object of the present invention to provide an implant design that will promote proper bony ingrowth from the body's natural healing process. It is still further an object of the present invention to provide an implant design having improved features to engage an insertion tool used in the spinal fusion process. It is still further an object of the present invention to provide a tapered leading edge to a cylindrical design to facilitate implant insertion. Yet still further, it is an object of the present invention provide an optimum design for ridges with optimum geometry.

### BRIEF SUMMARY OF THE INVENTION

The present invention specifically addresses and alleviates the above mentioned deficiencies associated with the prior art. More particularly, the present invention in a first aspect, is directed to an interbody fusion device comprising: a tapered head portion; a cylindrical body portion having an inner diameter and an outer diameter; and an end portion opposite the head portion, the end portion additionally comprising a disc shaped protrusion, the disc shaped protrusion having a carve out having a hexagonal shape, the carve out further including a threaded inner cavity for engaging a tool for insertion of said device.

In this aspect, the invention is additionally characterized in that the body portion further comprises a cavity about an axis aligned perpendicular to a longitudinal axis. Also, the body portion further comprises a plurality of anti-migration ridges including a “Z” shape having a peak followed by perpendicular ridge relative to a longitudinal axis, and followed by an angled constant slope to an adjacent peak. Further, the invention is characterized wherein the inner diameter corresponds to a width of the disc shaped protrusion.

In a preferred embodiment, the plurality of anti-migration ridges comprises no more and no fewer than four anti-migration ridges for optimum bony ingrowth and brittle fracture properties. Also according to a preferred embodiment, the disc protrusion further comprises two diametrically opposed cut-outs in the disc protrusion, the diametrically opposed cut-outs for further engaging an insertion tool. Further in this aspect, the invention is characterized wherein the tapered head portion further comprises a frustum conical shape however having rounded edges.

In yet another aspect, the invention is characterized as an interbody fusion device comprising: a tapered head portion; a cylindrical body portion having a first quarter portion, a second quarter portion, a third quarter portion, and a fourth quarter portion, wherein the first and third quarter portions comprise a cavity therethrough, and wherein the first and third quarter portions further comprise a plurality of anti-migration ridges, and wherein the second and fourth quarter portions comprise a smooth surface. An end portion is also provided opposite the head portion, the end portion configured to engage an insertion tool.

An additional embodiment for an end portion is characterized as comprising a hexagonal protrusion having a surface area for engaging the insertion tool; and a threaded cavity for further engaging the insertion tool.

These, as well as other advantages of the present invention will be more apparent from the following description and drawings. It is understood that changes in the specific structure shown and described may be made within the scope of the claims, without departing from the spirit of the invention.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The invention can be better visualized by turning now to the following drawings wherein like elements are referenced by like numerals.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1A is a perspective view of a first preferred interbody fusion device of the present invention;

FIG. 1B is a cross-sectional view of the interbody fusion device taken along sectional line 1B-1B in FIG. 1A;

FIG. 1C is an enlarged view of the area bound by line 1C in FIG. 1B;

FIG. 1D is a top plan view of the first preferred embodiment;

FIG. 1E is an end view illustrating an end portion of the present invention;

FIG. 1F is a profile view of the first invention embodiment;

FIG. 1G illustrates an interbody fusion device(s) in a disc space of a spine;

FIG. 1H is an additional illustration of an interbody fusion device (2) being employed between vertebrae;

FIG. 2A is a perspective view of a second preferred embodiment of the present invention;

FIG. 2B illustrates a cross-sectional view of the second embodiment taken along sectional line 2B-2B in FIG. 2A;

FIG. 2C is an end view of an end portion of the invention illustrated in relation to FIG. 2B;

FIG. 2D is a top plan view of the second preferred embodiment;

FIG. 2E is a profile view illustrating an end portion of the second preferred embodiment of the present invention;

FIG. 3A is a perspective view of another invention embodiment;

FIG. 3B is an end view thereof;

FIG. 4A is a perspective view of still another invention embodiment;

FIG. 4B is an end view thereof;

FIG. 5A is a perspective view of yet still another invention embodiment;

FIG. 5B is an end view thereof;

FIG. 6A is a perspective view of still further another invention embodiment;

FIG. 6B is an end view thereof;

FIG. 7A is a perspective view of an additional embodiment for engaging an insertion tool;

FIG. 7B is an end view thereof;

FIG. 7C is a perspective view showing a notch in addition to the diametrically opposed cut-outs for engaging a insertion tool;

FIG. 7D is an end view thereof;

FIG. 8A is a perspective view of an alternative design for a cylindrical body portion;

FIG. 8B is an top plan view thereof;

FIG. 9A is a perspective view of an alternative end portion of the present invention; and

FIG. 9B is an end view thereof.

### DESCRIPTION OF THE PREFERRED EMBODIMENTS

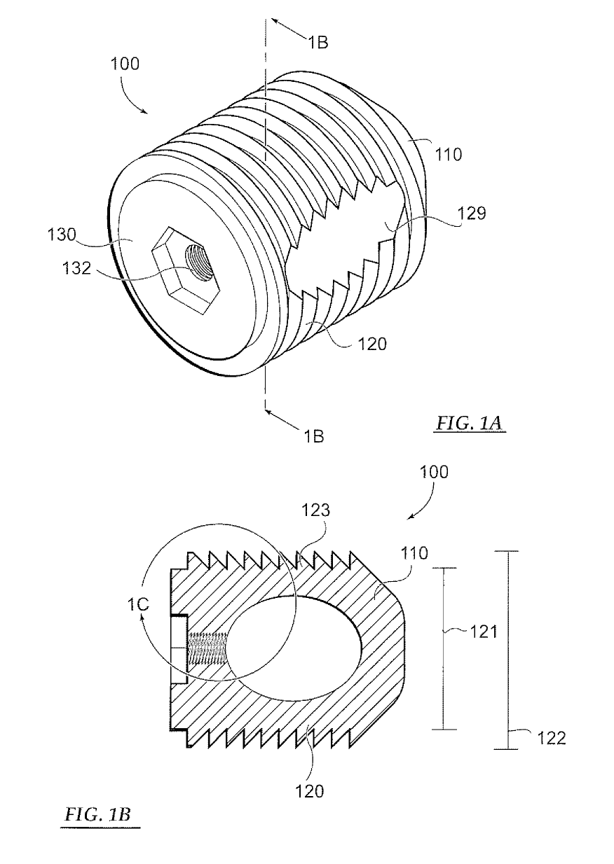

Referring initially to FIG. 1A, a perspective view of a first preferred embodiment of an interbody fusion device 100 is shown. This particular embodiment contains a cavity 129 that will naturally occur when a sample is obtained from femoral bone of an organ donor due to an existing canal within the femur. Coincidentally, the cavity 129 is beneficial to spinal fusion due to properties promoting bony ingrowth. Also importantly, when the device 100 is in place in the space formerly occupied by the intervertebral disc, the cavity should be aligned upward. Hence, the device 100 is actually illustrated on a side and accordingly FIG. 1D is described as the top plan view.

Also referring to FIG. 1B, a cross-sectional view of the invention embodiment 100 shown in FIG. 1A is provided. Herein, the device comprises a leading head portion 110 and a disc protrusion 130 at opposing ends. There between, a cylindrical body portion 120 has anti-migration ridges 123 as further detailed herein. The end portion 130 is configured to receive an insertion device; wherein according to this example 100, the end contains a hexagonal insert 131. At the end portion, the insertion device would be configured to engage the hexagonal insert 131. Referring to FIG. 1C, the insertion device will contain a threaded pin to matingly engage a threaded hole 132 of the present invention. Therefore for example, a fusion device 100 will not become unsecured from an insertion device when it is held upside down.

Also with regard to FIG. 1B and FIG. 1C, close inspection of the fusion device 100 reveals that it contains an inner 121 and an outer 122 diameter. The outer diameter 122 will correspond to peaks 124 of a plurality of ratcheted anti-migration ridges 123 or fins. The ridges 123 will help the fusion device 100 to remain in place as well as promote bony ingrowth. Generally, the device is cylindrical in shape and hence the outer diameter 122 is constant over the length of the device 100. The inner diameter 121 will correspond to a width of the end portion 130 and also to base points 124a on the plurality of ridges 123. Also generally, the ridges 123 are “Z” shaped having a peak 124 followed by a perpendicular ridge 125 (perpendicular to the length of the device 100, i.e. its longitudinal axis) to a base point 124a, and followed by an angled 126 constant slope to the next peak 123. In a preferred embodiment, the angle is 45°. In the example, eleven (11) peaks 123 are shown; however other designs with different peaks 123, 223 may be employed as further detailed herein. Since cavity 129 size is relatively constant, the present invention attempts to employ an optimum number of ridges 123, 223 so that ridges will not migrate forward with a reduced chance that expensive devices 100, 200, 300, 400, 500, 600, 700, 800, 900 will not fail due to brittle fracture during the insertion process. A design 100, 200 employing less ridges/peaks would make the ridges 123, 223 relatively thicker and more robust as further detailed herein. Also for optimum migration without fracture of the device 100, the last peak 127 closest to the end portion 130 comprises a different shape the having a flat summit 128 instead of a point 124.

The tapered head portion 110 provides a different shape than in prior art devices. The shape is essentially that of a truncated cone or frustum, i.e. a cone having an apex cut off by a parallel plane. However the truncated cone is further configured with a rounded tip to the frustum. The tapered head portion 110 will facilitate insertion of the spinal fusion device 100. FIG. 1E provides another view of an end portion 110 of the present invention showing hexagonal insert 131 with threaded hole 132.

FIG. 1D illustrates a top plan view of the present invention as the device 100 is typically inserted with the cavity 129 aligned upward, as illustrated in FIG. 1G, for example. With regard to FIG. 1F, a profile view of the invention embodiment 100 is illustrated. In this side aspect, the device 100 appears to have an hourglass shape due to the cavity 129. FIG. 1G illustrates a device inserted between adjacent vertebrae 911, 912 in the lumbar region of the spine, for example.

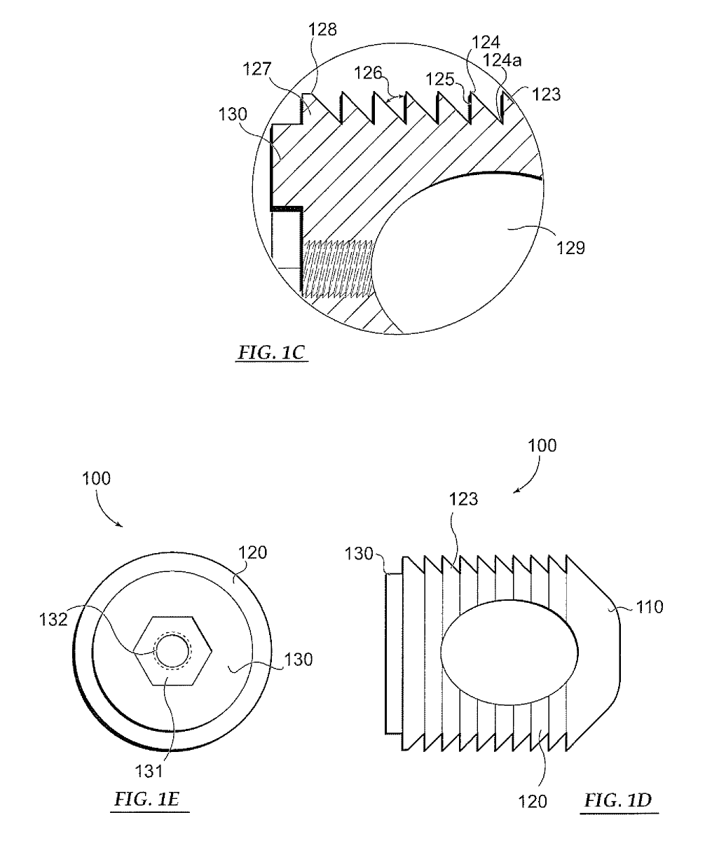

FIG. 2A through FIG. 2D illustrate an alternative embodiment 200 of the present invention. Similar to the first embodiment, the device comprises a tapered head portion 210 and a protruding end 230. As before, the end portion 230 has a hexagonal insert 231 and is configured to receive an insertion device. Importantly, this example comprises four (4) anti-migration ridges 223 as deemed to be an optimum number to prevent brittle fracture during insertion while additionally preventing post operative migration. Further as stated, cavity 229 has a fixed size. Additionally the invention herein 100, 200, 300, 400, 500, 600, 700, 800, 900 is devoid of threads to the cylindrical body portion so that impacting is only required and not torquing during insertion. This feature 123, 223 lowers a risk of failure due to fracture, and further eliminates one step in the insertion method. Similar to the first preferred embodiment 100, the ridge 227 closest to the end portion 230 has a flat summit 228 to facilitate insertion and to prevent migration thereafter.

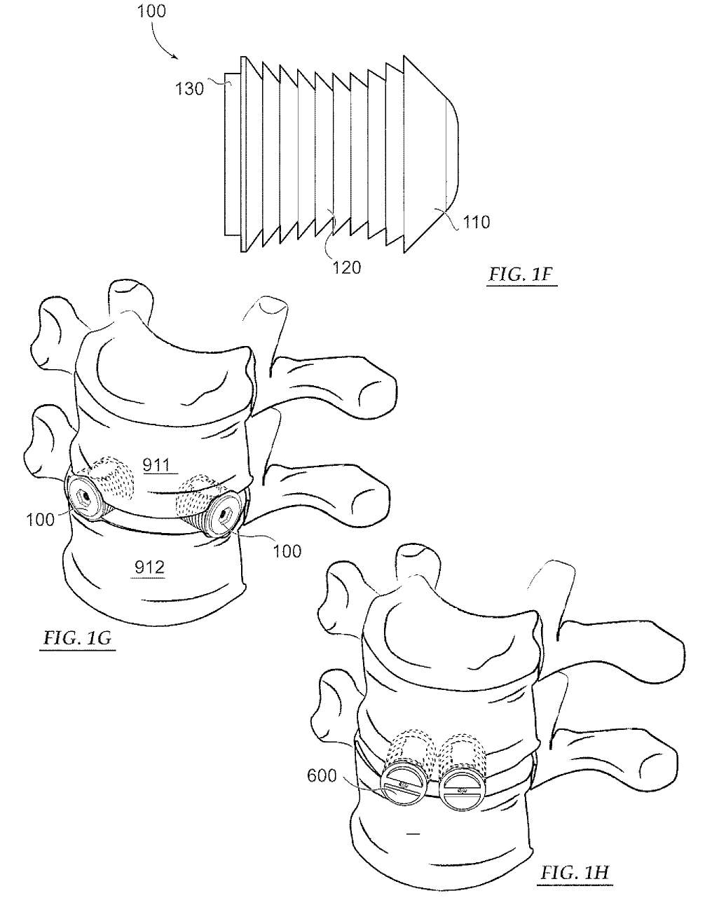

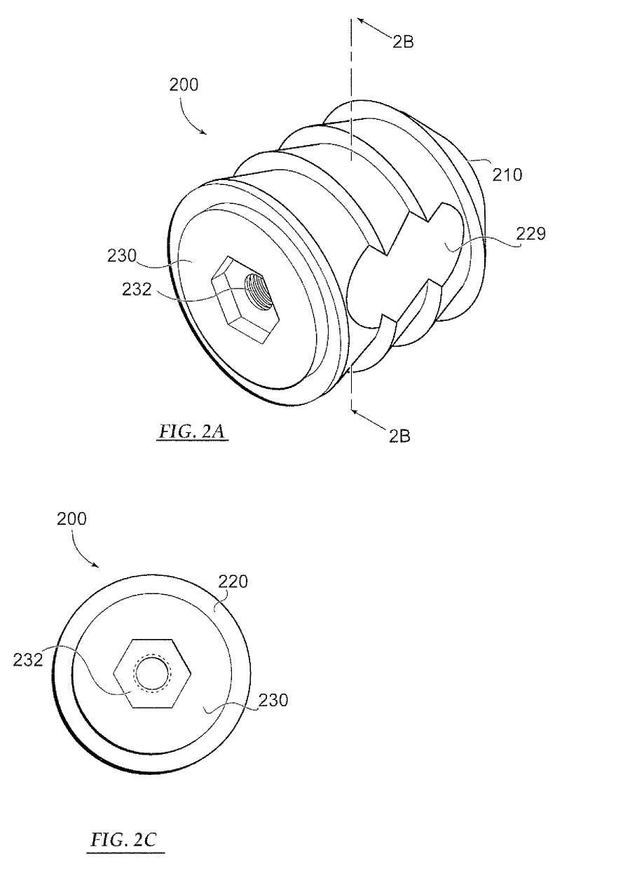

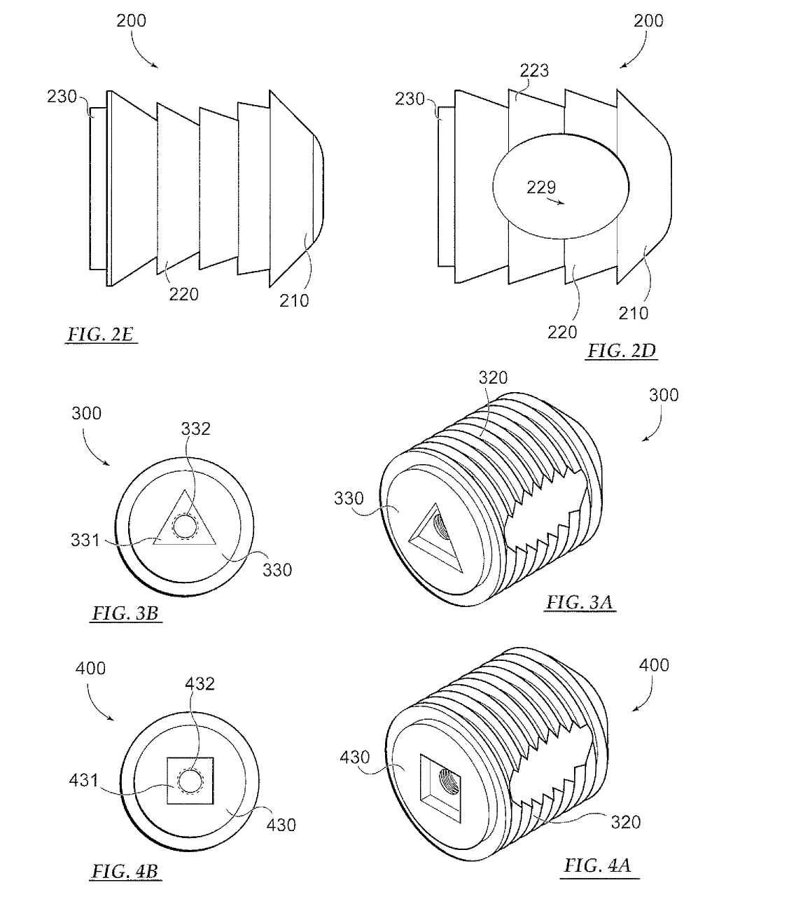

FIG. 3A and FIG. 3B illustrate an alternative triangle insert 331 for engagement for an insertion tool. Similarly, FIG. 4A, FIG. 4B, FIG. 5A, and FIG. 5B show square 431 and rectangle 531 inserts respectively. As shown, body portions 320, 420, 520, are coupled to ends 330, 430, 530 having female inserts 331, 431, 531, with female threaded cavities 332, 432, 532.

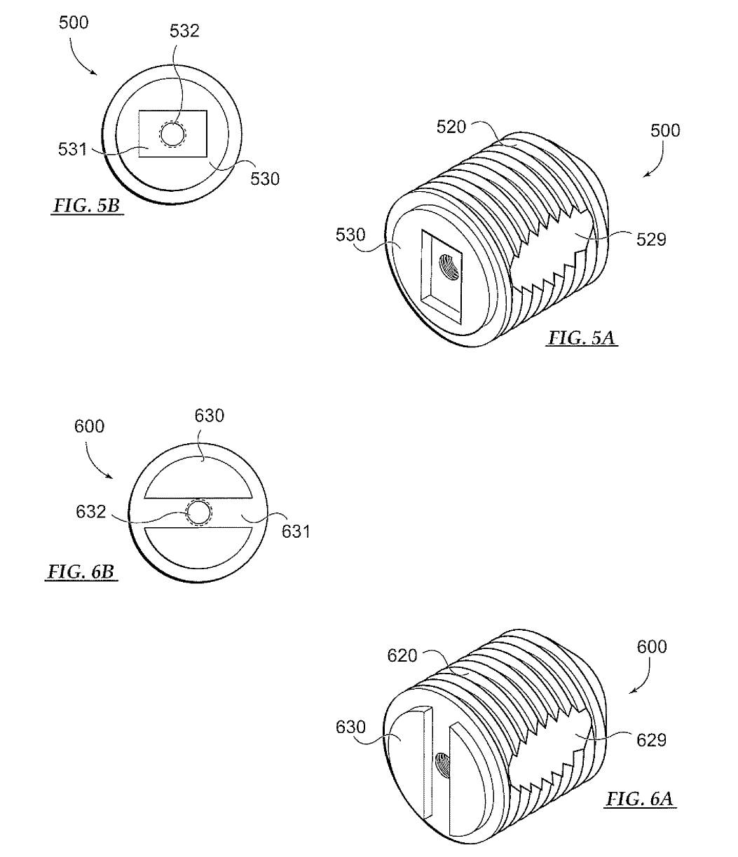

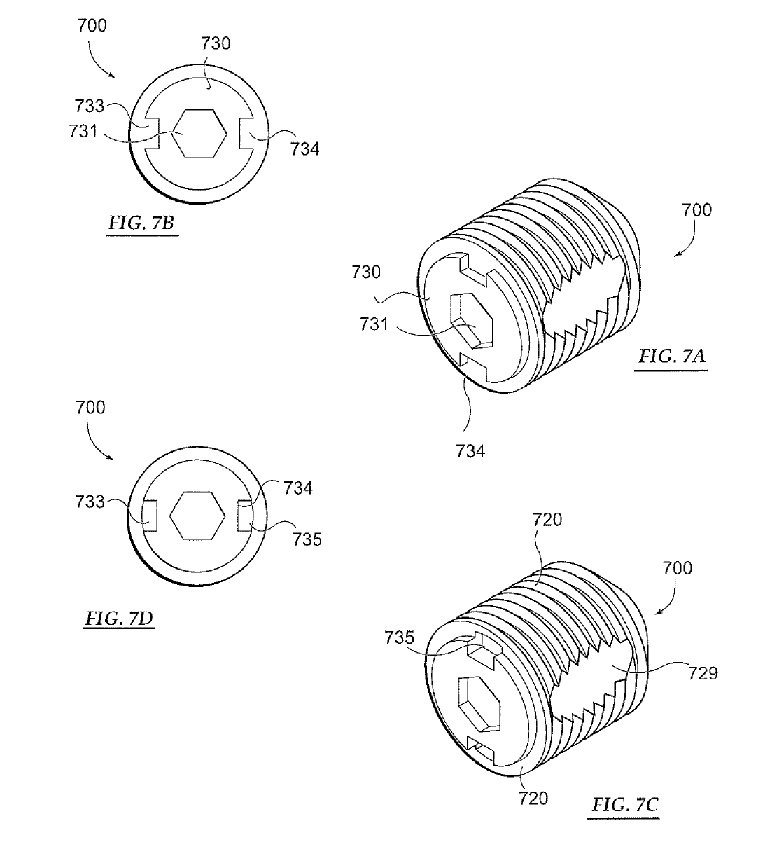

In the FIG. 6A and FIG. 6B example 600, a cylindrical body portion 620 has a protruding end 630 as stated herein. However, in this embodiment 600, a flat head screwdriver insert 631 is provided. For this, the insertion device would have a working end resembling a flat head screw driver however modified with a threaded pin as discussed herein. The configuration includes a slot 631 to receive the flat head portion of the insertion device. As shown, the slot 631 is aligned perpendicular to the cavity 629 (FIG. 6A). If the slot 631 were alternatively aligned with the cavity, it would result in a relatively weak area in the material between the cavity 629 and the slot 631. Similarly, it should be appreciated that the long axis of the insert 531 in FIG. 5A should be aligned perpendicular to its cavity 529; and further insert 731 with diametrically opposed cut-outs 733, 734 (FIG. 7A and FIG. 7B) are aligned perpendicular to its cavity 729 as detailed herein.

With regard to FIG. 7A and FIG. 7B, an additional embodiment 700 of an end portion 730 for engaging an insertion tool is provided. In this example, the threaded cavity 132, 232, 332, 432, 532, 632, 932 is absent and replaced with a pair of diametrically opposed cut-outs 733, 734. The diametrically opposed cut-outs 733, 734 together with a hexagonal insert 731 provide a friction fit to an insertion tool. In a preferred embodiment, the diametrically opposed cut-outs 733, 734 will further each have a notch 735 that extends a few millimeters into the body portion 720.

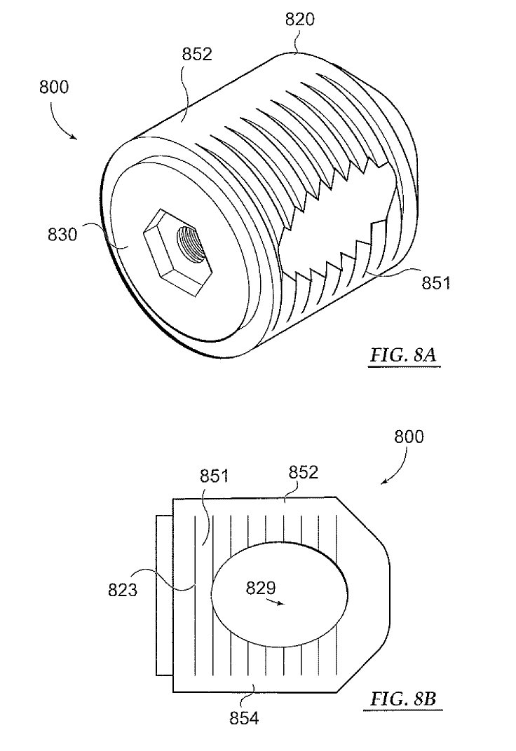

Now turning to FIG. 8A and FIG. 8B an additional embodiment 800 to a cylindrical body portion 820 is provided. Initially, it should be appreciated that cavity 829 is aligned upward in use, so the illustration in FIG. 5A depicts a device lying on its 800 side. Therefore, FIG. 8B is a top plan view. Herein, cylindrical body portion 820 comprises four quarter portions, namely first 851 second 852 third (not shown, however identical to first 851 quarter portion) and fourth 853 quarter portions. The first 851 and third quarter portions comprise the anti-migration ridges 823 carved out of a smooth surface 852, 854, since these portions (851, 853) will have the most direct contact with adjacent vertebrae 911, 912 (FIG. 1G). Second 852 and fourth 854 quarter portions will comprise a smooth surface without ridges 823, for enhanced strength of the device 800, as desired by a user.

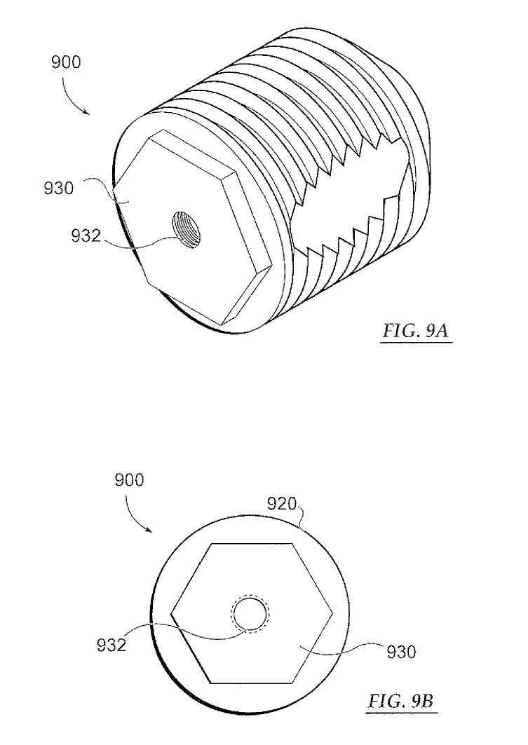

With regard to FIG. 9A and FIG. 9B, yet another alternative 900 for an end portion 930 for engaging an insertion tool is provided. Whereas previous embodiments provided female-female 131, 132 connection, this embodiment is female-male 930, 932. Hence, an insertion tool can fit over the hexagonal protrusion 930 and fit into the threaded cavity 932. This option 900, 930 provides more surface area 930 for rotation in an event that rotational adjustment is needed.

The invention also provides novel processes and method for fusion of vertebrae 911, 912. The method comprises proving a cadaver femoral bone; machining a tool to cut a spinal fusion device having a proper geometry; and freeze drying the device so it can be preserved and stored for when it's needed without having to rely on freezer storage and dry ice for transportation. Next proper space is maintained in the area between the vertebrae 911, 912 to prepare for the insertion process. Next, the device is impacted into place and not torqued or screwed.

Many alterations and modifications may be made by those having ordinary skill in the art without departing from the spirit and scope of the invention. Therefore, it must be understood that the illustrated embodiments have been set forth only for the purposes of example and that it should not be taken as limiting the invention as defined by the following claims. For example, notwithstanding the fact that the elements of a claim are set forth below in a certain combination, it must be expressly understood that the invention includes other combinations of fewer, more or different elements, which are disclosed in above even when not initially claimed in such combinations.

While the particular Interbody Fusion Device as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

What is claimed is:

1. An interbody fusion device comprising:
a tapered head portion;
a cylindrical body portion having an inner diameter and an outer diameter; and
an end portion opposite the head portion, the end portion additionally comprising a disc shaped protrusion, the disc shaped protrusion having a carve out having a hexagonal shape, the carve out further including a threaded inner cavity for engaging a tool for insertion of said device.

2. The interbody fusion device of claim 1, the body portion further comprising a cavity about an axis aligned perpendicular to a longitudinal axis.

3. The interbody fusion device of claim 1, the body portion further comprising a plurality of anti-migration ridges comprising a “Z” shape having a peak followed by perpendicular ridge relative to a longitudinal axis, and followed by an angled constant slope to an adjacent peak.

4. The interbody fusion device of claim 1 wherein the inner diameter corresponds to a width of the disc shaped protrusion.

5. The interbody fusion device of claim 3, the plurality of anti-migration ridges comprising no more and no fewer than four anti-migration ridges for optimum bony ingrowth and brittle fracture properties.

6. The interbody fusion device of claim 1, the disc protrusion further comprising two diametrically opposed cut-outs in the disc protrusion, the diametrically opposed cut-outs for further engaging an insertion tool.

7. The interbody fusion device of claim 1, the tapered head portion further comprising a frustum conical shape however having rounded edges.

8. An interbody fusion device comprising:
a tapered head portion;
a cylindrical body portion having an inner diameter and an outer diameter, the cylindrical body portion further comprising a plurality of anti-migration ridges, wherein a majority of the plurality anti-migration ridges comprises a “Z” shape having a peak followed by perpendicular ridge relative to a longitudinal axis, and followed by an angled constant slope to an adjacent peak, and wherein a trailing anti-migration ridge comprises a flat summit; and
an end portion opposite the head portion configured for engaging a tool for insertion of said device.

9. The interbody fusion device of claim 8, the end portion additionally comprising a disc protrusion, wherein the disc protrusion protrudes with respect to the trailing anti-migration ridge, the disc having a carve out for the engaging a tool for insertion of said device.

10. The interbody fusion device of claim 9 wherein the carve out is chosen from a group consisting of a triangular carve out, a square carve out and a rectangular carve out.

11. The interbody fusion device of claim 9 wherein the disc protrusion comprises a hexagonal carve out about a center thereof, and wherein the disc protrusion further comprises a pair of diametrically opposed cut-outs in an outer edge of the disc protrusion, the diametrically opposed cut-outs for further engaging an insertion tool.

12. The interbody fusion device of claim 9 wherein the disc protrusion comprises a slot to receive a flat head portion of the insertion device, and wherein the cylindrical body portion comprises a cavity about an axis aligned perpendicular to a longitudinal axis and wherein the slot is aligned perpendicular to the cavity.

13. The interbody fusion device of claim 8, wherein the “Z” shape comprises a 45 degree angle.

14. The interbody fusion device of claim 8 wherein the inner diameter corresponds to a width of the disc shaped protrusion.

15. The interbody fusion device of claim 3, the majority of the plurality of anti-migration ridges comprises three (3) anti-migration ridges.

16. The interbody fusion device of claim 9, the disc protrusion further comprising two diametrically opposed cut-outs in the disc protrusion, the diametrically opposed cut-outs for further engaging and insertion tool.

17. An interbody fusion device comprising:
a tapered head portion;
a cylindrical body portion having a first quarter portion, a second quarter portion, a third quarter portion, and a fourth quarter portion, wherein the first and third quarter portions comprise a cavity therethrough, and wherein the first and third quarter portions further comprise a plurality of anti-migration ridges, and wherein the second and fourth quarter portions comprise a smooth surface; and
an end portion opposite the head portion, the end portion configured to engage an insertion tool.

18. The interbody fusion device of claim 17, the end portion comprising:
a hexagonal protrusion having a surface area for engaging the insertion tool; and
a threaded cavity for further engaging the insertion tool.

19. The interbody fusion device of claim 17, the tapered head portion having a frustum conical shape however having rounded edges.
